(19) United States
(12) Patent Application Publication
(10) Pub. No.: US 2025/0241521 A1
(43) Pub. Date: Jul. 31, 2025

(54) MAGNETIC HANDPIECE FOR AN ENDOSCOPIC GUIDE, IN PARTICULAR FOR COLONOSCOPES, AND ENDOSCOPY SYSTEM COMPRISING SAID HANDPIECE

(71) Applicant: ENDOSTART S.R.L., Certaldo, Firenze (IT)

(72) Inventors: Alessandro TOZZI, Certaldo, Firenze (IT); Tommaso TUCI, Certaldo, Firenze (IT); Fabiana MASIELLO, Certaldo, Firenze (IT); Giorgia MONDELLO, Certaldo, Firenze (IT); Denise LAZZERI, Certaldo, Firenze (IT)

(21) Appl. No.: 18/855,311

(22) PCT Filed: Mar. 28, 2023
(86) PCT No.: PCT/IB2023/053081

§ 371 Date: Oct. 8, 2024

(30) Foreign Application Priority Data
Apr. 14, 2022 (IT) 102022000007475

(51) Int. Cl. A61B 1/00; A61M 25/10

(52) U.S. Cl. CPC A61B 1/00158; A61B 1/00066; A61B 1/00082; A61M 25/10182

(57) ABSTRACT

A magnetic handpiece for an endoscopic guide is for application in colonoscopy. A magnetic handpiece system includes the magnetic handpiece and a casing. The magnetic handpiece includes a magnetic field source having one or more permanent magnets, and a hollow body having a longitudinal axis, a first end and a second end. A movable support element of the magnetic source is movably accommodated in the hollow body through a screw coupling. The movable support element is movable along the axis between a raised, or non-operating, position and lowered operating positions.

### CROSS-REFERENCE TO RELATED APPLICATIONS

The present application is a National Stage Filing of PCT International Application No. PCT/IB2023/053081 filed on Mar. 28, 2023, which claims priority to Italian application No. 102022000007475, filed with the Italian Patent Office on Apr. 14, 2022, which applications are incorporated herein by reference in their entirety.

### FIELD OF THE INVENTION

The present invention relates to a magnetic handpiece for an endoscopic guide for catheters or endoscopes, particularly suitable for application in colonoscopy.

Examples of such instruments are colonoscope, enteroscope, gastroscope, duodenoscope, echoendoscope, and the like.

### BACKGROUND ART

Endoscopic guides (or “guide wires” in technical jargon) are used in the field to position and act as a guide, in particular for a catheter, which is normally mounted sliding thereon; in this regard, the catheter is provided with an operating channel in which the guide itself is placed in assembled condition.

Various types of endoscopic guides are known from the prior art, according to the applications they are intended for.

A type of endoscopic guide provides an anchoring head which allows the guide to remain in place and act as a support for the operations of the catheter and endoscope.

In this regard, in certain embodiments the guides are provided with an inflatable balloon which, once introduced into the lumen of the organ and positioned, is inflated with air up to interfering with the walls of the lumen itself, thus remaining blocked in place.

Although these guides with an expandable anchoring head are used, they have considerable drawbacks.

Such anchoring heads are difficult to use in certain fields of endoscopy, such as in the case of colonoscopy, for example, due to the anatomical shape of the organ in which the guide is introduced. Indeed, the diameter of the lumen in the colon can vary from 2-3 cm up to 6-8 cm in relation to the segment considered and the presence or lack of pathological conditions or anatomical anomalies. This would result in the need for even quite bulky balloons.

Moreover, the anchoring via the balloons is obtained by virtue of the friction generated between the balloon and the surface of the organ mucosa. This may result in lesions to the organ walls. In the case of colonoscopy, the friction blockage results in the need to increase the pressure on the wall of the viscera, stretching out the balloon more, to obtain more stable anchoring. However, this operation could result in the excessive stretching of the viscera, with the risk of causing pain or even laceration of the tissue. Moreover, at times, especially in the elderly or in certain pathological conditions, the viscera can be dilated or flaccid, to the point of providing no resistance to the balloon dilation. In this condition, not only can the stretching of the balloon be risky, but also ineffective.

However, obtaining a firm and stable anchoring is necessary in order to ensure that the guide can also operate for colonoscopes and endoscopes in general.

In particular, the colonoscopy technique requires care in introducing the colonoscope up to the end point of the colon because the colon has a winding path and flaccid walls, whereby loops preventing the progression of the colonoscope tip and stretching the walls of the colon are formed, resulting in the occurrence of pain and the risk of laceration of the viscera: in these cases, the physician must maneuver the instrument in an adequate manner so as to rectify the path as much as possible and proceed by introducing the colonoscope up to the end point.

Many problems associated with the devices of the prior art have been overcome by an endoscopic guide and an endoscopy system comprising it as described in European Patent EP3399901A1 to the Applicant. Such a patent describes a system for magnetically anchoring the tip of the endoscopy guide by introducing a magnetic fluid consisting of an aqueous suspension of iron powder, magnetite, maghemite or ferrofluid into an inflatable balloon. The anchoring is obtained by applying, from the outside, a magnetic field having adequate force by means of a specific handpiece.

Although such a system allows obtaining an adequate anchoring to the wall of the colon without obstructing the lumen of the organ and without damaging the tissue thereof, it has various drawbacks.

In particular, as for the magnetic handpiece here described, it is the result of complex and costly manufacturing, has an excessive weight and is difficult to shield both during use and when the handpiece is stored. An inadequate shielding of the magnetic field has two disadvantages: giving rise to possible interference with other devices placed nearby and scattering the magnetic field in different directions from the preferential one of interacting with the balloon filled with magnetic fluid. Thus, it is a task of the invention to provide a magnetic handpiece system which solves one or more of the technical problems set out above with respect to the known magnetic handpiece.

In particular, it is an object of the invention to manufacture a magnetic handpiece system which is both quicker and simpler to operate and has smaller size and weight, while being relatively affordable and simple to manufacture.

### SUMMARY OF THE INVENTION

One or more of the tasks and objects set out above and others which can be apparent below are achieved by:
1) a magnetic handpiece system, comprising a magnetic handpiece and a casing, where the magnetic handpiece comprises a magnetic field source consisting of one or more permanent magnets and where the magnetic handpiece comprises a hollow body having a longitudinal axis, a first end and a second end, in which a movable support element of the magnetic source is movably accommodated through a screw coupling, where the movable support element is movable along the longitudinal axis between a raised, or non-operating, position and a plurality of lowered operating positions.

Other objects of the invention are:
2) the system as in point 1), where the body comprises a closing element at the top which comprises a central sleeve portion having an axial opening in which a shaft is inserted, an inner end of the shaft being connected to the movable support element and an outer end of the shaft being connected to a handle, the handle preferably having a spiral shape, for example in clockwise direction, to make the correct rotation direction more intuitive for screwing and unscrewing the movable support element and thus increasing or decreasing the magnetic attraction force applied;
3) the system as in point 1) or 2), where the movable support element comprises a hollow cylindrical portion comprising an outer thread and an inner surface comprising a plurality of ribs, the hollow cylindrical portion defining a hollow interior configured to house the magnetic source; and where the body comprises in turn, on an inner surface thereof, a thread which is couplable to the thread of the movable support element;
4) the system as in any one of points 1) to 3), where the magnetic source has a magnetization equivalent to a discoidal permanent magnet with N52 magnetization having a diameter between 70 and 90 mm, a thickness between 30 and 60 mm, and a weight between 1.81and 2.8 kg, and is selected from:
a) a cylinder-shaped magnet having axial magnetization;
b) a Halbach Array-type configuration comprising a plurality of magnets, preferably eight magnets, arranged in a crown about a cylinder-shaped central magnet;
c) a Halbach Array-type configuration comprising four magnets arranged along the sides of a parallelepiped central magnet, i.e., peripheral with respect to said central magnet;
d) a Halbach Array-type configuration comprising two magnets arranged sandwiched along two sides of a parallelepiped central magnet, where:
in configuration b), the central magnet is placed with axial polarization, while the plurality of magnets in a crown has a polarization arranged along the rays converging towards the longitudinal axis of the central magnet,
in configuration c), the central magnet is placed with axial magnetization, while the four peripheral magnets have a magnetization parallel to a plane perpendicular to the magnetization
in configuration d), the central magnet and the two sandwiched magnets are placed with magnetization along directions at 90° from each other;
5) the system as in point 4), where:
the magnetic source according to configuration a) consists of a magnet having dimensions of about D85-86 mm×Sp45-45.5 mm, where the total weight of the magnetic source is about 2 kg:
the magnetic source according to configuration b) consists of a central magnet having a diameter of about 74 mm and a thickness of about 35 mm, and of eight parallelepiped magnets having dimensions of about Lc15×Lu26×Sp35 mm, where the total weight of the magnetic source is about 2 kg, and where the eight magnets in a crown are spaced apart from the central magnet by about 1 mm;
the magnetic source according to configuration c) consists of a central magnet having dimensions of Lc=Lu60-61 mm×Sp35-44 mm, and of four peripheral magnets having dimensions of Lc15-16×Lu60-61×Sp35-44 mm, where the total weight of the magnetic source is between 2 and 2.5 kg;
the magnetic source according to configuration d) consists of a central magnet having dimensions of about Lc44×Lu45×Sp58 mm, and of two sandwiched magnets having dimensions of about Lc28-16×Lu45×Sp58 mm, where the total weight of the magnetic source is about 2 kg,
where Lc=short side, Lu=long side, Sp=thickness, and D is the diameter of the base of the cylinder;
6) the system as in any one of points 1) to 5), where the magnetic handpiece comprises magnetic field concentrator elements obtained using ferromagnetic material shields arranged along the walls of the movable support element or the body and optionally above the magnetic source, in the form of a plurality of plates or as a continuous or discontinuous annular band, and can consist of a single sheet of ferromagnetic material or a plurality of sheets, placed in contact or spaced apart;
7) the system as in point 6), where said ferromagnetic material is a metal alloy comprising 76-80%, or about 80%, nickel, about 14.5-15% iron, 3-6%, or about 5%, molybdenum, 4-55% copper, and traces of silicon;
8) the system as in any one of points 1) to 7), where the casing comprises a containment shell placed on a stand comprising a base and a pole, where the base preferably comprises wheels, the containment shell comprising a basket and a lid provided with a handle, the lid being hinged on the basket at two hinges placed in diametrically opposite points, and where the lid and optionally the basket as well are shaped like a spherical cap so that the containment shell, as a whole, is substantially spherical or semi-ovoidal in shape;
9) the system as in point 8), where the basket comprises a toroidal body which is preferably internally empty so as to create a space, the toroidal body comprising a central seat sized to house the magnetic handpiece;
10) the system as in point 8) or 9), where a catch comprising a tilting bar hinged in a median point thereof on a ridge placed on the surface is placed on an outer surface of the basket, in a position close to an upper edge, a first and a second retainer tooth being placed close to two ends of the bar, where the first retainer tooth is configured to snappingly couple in a seat recessed in the surface of the basket, while the second retainer tooth is configured to be coupled to a ridge placed along an edge of the lid, in a median position with respect to the hinges;
11) the system as in any one of points 8) to 10), where the basket comprises magnetic field shielding elements consisting of ferromagnetic material plates, preferably a metal alloy comprising 76-80%, or about 80%, nickel, about 14.5-15% iron, 3-6%, or about 5%, molybdenum, 4-55% copper, and traces of silicon, the overall thickness of the shielding elements being preferably between 3 mm and 6 mm;
12) the system as in point 11), where the shielding elements comprise an annular sheet and a plurality of U-shaped sheets configured so as to also cover the bottom of the basket, the shielding elements being preferably accommodated in the space inside the toroidal body;
13) a magnetic handpiece as defined in any one of points 1) to 7);
14) a casing for a magnetic handpiece as defined in any one of points 8) to 12);
15) an endoscopy kit, comprising:
a magnetic handpiece as defined in any one of points 1) to 7),
an endoscopic guide comprising a tubular guide element and an anchoring head, where the tubular guide element comprises a longitudinal cavity, the anchoring head being provided with an expandable container configured to accommodate a ferromagnetic agent,
a pre-filled syringe filled with an aqueous suspension of a preset amount of ferromagnetic agent; or a glass vial filled with the powdered ferromagnetic agent, preferably carbonyl iron, and a pre-filled syringe filled with saline solution (aqueous solution of NaCl and/or sodium citrate); or a glass vial with the ferromagnetic agent suspension, preferably carbonyl iron, and an empty syringe.

The invention further relates to an endoscopy system, preferably for colonoscopy, comprising an endoscopic guide acting in conjunction with an external magnetic field source, where the magnetic field source is associated with a manually maneuverable device, i.e., also without movement apparatuses or assisted support, where the manually maneuverable device is a magnetic handpiece in which the magnetic field source is movable between an operating position and a safety or resting position, where:
in the operating position, the magnetic field source is close to the operating end of the handpiece,
in the safety or resting position, the magnetic field source is far from the operating end of the handpiece.

Further features and advantages will become apparent from the description of preferred, but not exclusive, embodiments of the invention, illustrated by way of non-limiting example.

### BRIEF DESCRIPTION OF THE DRAWINGS

FIG. 1 is a side view of the magnetic handpiece according to the invention;

FIG. 2 is a sectional view of the magnetic handpiece according to direction I-I in FIG. 1;

FIG. 3 is a top view of the handle of the magnetic handpiece in FIG. 1;

FIG. 4 is a bottom perspective view of a detail of the magnetic handpiece in FIG. 1;

FIGS. 5A and 5B depict perspective and plan views, respectively, of a first configuration of magnets according to the invention;

FIGS. 6A and 6B depict perspective and plan views, respectively, of a second configuration of magnets according to the invention;

FIGS. 7A and 7B depict perspective and plan views, respectively, of a third configuration of magnets according to the invention;

FIGS. 8A and 8B depict perspective and plan views, respectively, of a fourth configuration of magnets according to the invention;

FIG. 9 is a diagrammatic depiction of the lines of force of the magnetic field with and without side shielding;

FIG. 10 is a side view of the handpiece in FIG. 1 showing, in transparency, a system for concentrating the magnetic field;

FIG. 11 is a side view of a casing of the magnetic handpiece in FIG. 1;

FIG. 12 is a sectional side view of the casing in FIG. 11 with the magnetic handpiece of the invention therein;

FIG. 13 shows a sectional side view of a detail of the casing in FIG. 11;

FIG. 14 shows a top sectional view of a detail in FIG. 13;

FIG. 15 shows a perspective view of a different detail of the casing in FIG. 11;

FIG. 16 shows a perspective sectional view of the detail in FIG. 13 with the magnetic handpiece in FIG. 1 therein;

FIG. 17 shows a side view of a different detail of the casing in FIG. 11;

FIG. 18 shows a perspective view of a variant of the casing of the magnetic handpiece.

### DETAILED DESCRIPTION OF THE INVENTION

Firstly, it is useful to describe how a diagnostic colonoscopy examination is performed to better understand the invention.

The colonoscope is a flexible endoscope characterized by a tip (about 15 cm long), a body (about 130 cm long), and a manipulator. The tip is movable and accommodates the camera, the lights and the orifice of the operating channel therein. The tip can be maneuvered by moving the knobs located on the manipulator. The body has a flexibility which, in certain models, can also be selected for performing certain endoscopic maneuvers. The cables for transmitting the images, the electric supply and the tie rods for connecting the tip to the knobs slide inside the body.

In addition to the knobs for orienting the tip, the manipulator is also characterized by the external orifice of the operating channel and by the controls for drawing or blowing air and recording images or videos.

The diagnostic colonoscopy aims to inspect the whole surface of the colon mucosa so as to identify any pathological alterations thereof which possibly require further diagnostic or therapeutic interventions. It is necessary for the colonoscope tip provided with cameras and lights to reach the last part of the colon, referred to as the caecum. If indeed the instrument were not to reach the caecum, the physician could not affirm whether there are any pathological alterations in the unexplored segment.

The positioning of the colonoscope is performed by an endoscopic guide which is moved forward into the lumen of the colonoscope up to stretching out from the distal end thereof and advancing as deeply as possible, to then guide the path of the colonoscope. A similar technique is performed to introduce other endoscopes, as well as to introduce catheters into the vein system.

The colon is a movable hollow, tube-like organ with loose walls. Unlike other hollow tube-like organs, such as arteries, for example, it has a larger diameter, the walls can stretch out over several centimeters, and in certain segments, the viscera can be mobilized in the abdominal cavity, at times by up to 30 cm.

Such anatomical features prevent the insertion of catheters or guides over segments of more than 10-30 cm. Indeed, once inserted into the rectum via the anus, catheters tend to coil up on themselves due to the large diameter of the lumen and the curves of the viscera. Therefore, unlike what is done in the vascular field, the catheterization of the colon is difficult to perform, unless over a short segment. A catheter or an endoscopic guide do not have adequate mobility of the tip or adequate bearing strength even during a radiological checkup. Bearing strength of an endoscope, catheter, or endoscopic guide means the capacity to transmit the thrust from the body of the instrument to the tip thereof. The greater the rigidity of an endoscope, the greater the bearing strength. However, a too rigid endoscope cannot be easily maneuvered along the winding path of the colon. The colonoscope is designed so as to be a compromise between adequate flexibility and adequate bearing strength.

However, bearing strength progressively decreases if the colonoscope is in an excessively flexed arrangement. For example, if the colonoscope forms a loop during the colonoscopy, it can no longer be capable of transmitting the thrust from the body to the tip, and therefore the endoscopist is not able to further advance the instrument and the camera thereof. In these cases, the endoscopist manually pushing the instrument only results in the transmission of the force on the walls of the viscera, resulting in pain and the risk of trauma, moreover without advancing the tip.

In order to solve this drawback, the endoscopic guide suggested in the above-mentioned Patent EP3399901A1 to the Applicant allows performing a method for introducing the colonoscope comprising the following steps:
introducing, via the patient's anus, the distal end of the colonoscope up to reaching a blocking point, i.e., a point beyond which the colonoscope can no longer be advanced;
advancing the endoscopic guide up to a distance of 4-10 cm past the distal end of the colonoscope;
introducing, into the expandable container of the endoscopic guide, a magnetic or ferromagnetic agent so that the container reaches an expanded condition;
applying an external magnetic field by means of a magnetic field source at the area of the abdomen in which there is the expanded container with the magnetic or ferromagnetic agent so as to attract the container against an anchoring point on the wall of the colon with a retaining force on the magnetic or ferromagnetic agent, with respect to a transverse pulling direction, at least equal to or greater than 5 Newton;
tensioning the endoscopic guide by applying a pulling force of at least 5 Newton on the proximal end of the endoscopic guide;
advancing the colonoscope up to the anchoring point of the container;
repeating the above steps up to reaching an anchoring point at the caecum.

Pulling the endoscopic guide aims to keep the guide tensioned, allowing an easier sliding of the endoscope, and to at least partially reduce the curvature of the loops of the colon, even in this case in order to promote the introduction of a relatively rigid endoscope.

As mentioned above, when performing the above-described method, the endoscopy system described in EP3399901A1 has underlined certain criticalities which have made the use thereof poorly applicable to a diagnosis. Thus, the present invention results from the need to overcome such criticalities.

The endoscopy system with which the magnetic handpiece system according to the invention acts in conjunction, comprises an endoscopic guide, in particular for colonoscopes, comprising a tubular guide element and an anchoring head, where the tubular guide element comprises a longitudinal cavity, the anchoring head being provided with an expandable container configured to accommodate a ferromagnetic agent, the expandable container being in communication with said longitudinal cavity, where the endoscopy system further comprises a ferromagnetic agent configured to be movable in said longitudinal cavity to fill/empty the expandable container.

The expandable container is manually expandable and retractable, preferably in an elastic manner, so as to switch from a retracted condition, in which it substantially adheres to the body of the tubular guide element, to an expanded condition, in which it projects externally from the body of the tubular guide element, and vice versa; the filling or emptying of the expandable container by means of the ferromagnetic agent results in the switching from the retracted position to the expanded one, and vice versa.

The expandable container comprises, or alternatively consists of, an inflatable balloon. Examples of inflatable balloons are those normally used in endoscopy, for example nylon balloons or balloons made of a polyether-polyamide block copolymer, or a compound thereof with a polyamide. Such copolymers and the compounds thereof with polyamides are known and were described in detail in Patent Publication WO 2007/132485 A1.

In preferred embodiments, the expandable container is sized so as to have a capacity of 22-28 ml, more preferably of about 25 ml, and when in expanded condition, a length between 55 and 65 mm, more preferably of about 60 mm, and a transversal diameter between 20 and 30 mm, more preferably of about 25 mm. Such dimensions allow not occluding the lumen of the colon, which has a varying diameter between 3 and 7 cm, depending on the cross section observed.

The ferromagnetic agent most suited to the purposes of the invention is an aqueous suspension of so-called carbonyl iron, although so-called ferrofluid can be used, or a suspension of magnetite or maghemite.

Carbonyl iron is a highly pure iron (97.5% for grade S, at least 99.5% for grade R), prepared by chemical decomposition of purified iron pentacarbonyl. It usually has the aspect of a grey powder consisting of spherical micro particles. Most of the impurities are carbon, oxygen and nitrogen. The most common uses are in powder metallurgy, metal injection molding and in various special products. Carbonyl iron powder is used in pharmaceutical applications to treat iron deficiency and as an iron food supplement due to the low toxicity thereof.

For the purposes of the present invention, carbonyl iron preferably having the following particle size profile is used:
D10=3-4.5 microns, more preferably about 4.1 microns
D50=5-10 microns, more preferably about 9.5 microns
D90=4-30 microns, more preferably about 26 microns

The particle size of the ferromagnetic particles can be determined with known methods, such as:
direct observation by electronic microscopy;
determination with methods of “laser light scattering” on the aqueous suspension using a particle size laser analyzer (Malvern Mastersizer 3000) with LALLS (Low Angle Laser Light Scattering) technique using the Fraunhofer calculation theory.

The aqueous suspension of carbonyl iron according to the invention preferably is a suspension in purified water in which the carbonyl iron is present in an amount by weight between 45% and 60%, preferably between 50% and 55%, more preferably between 52% and 53%, with respect to the total weight of the suspension. Preferably, said aqueous suspension further comprises tribasic sodium citrate in amounts by weight between 2.5% and 4.5% with respect to the total weight of the suspension, and/or sodium chloride in amounts by weight between 5% and 8% with respect to the total weight of the suspension.

The tribasic sodium citrate promotes the dispersion of the particles of carbonyl iron in the suspension.

The sodium chloride serves to inhibit bacterial proliferation.

The aqueous suspension of carbonyl iron is preferably degassed so as to avoid oxidative processes of the iron by the oxygen absorbed in water.

It has been noted that the use of the aqueous suspension of carbonyl iron according to the present invention results in several advantages as compared to the known solutions, i.e.:
i) it has an increased magnetic response, whereby the quantity of aqueous suspension used, and therefore the filling volume of the expandable container 4, can be decreased;
ii) the spherical shape of the particles results in less viscosity of the aqueous suspension and reduces the risk of the formation of clusters and/or masses;
iii) carbonyl iron is widely available on the market, has a moderate cost and a low toxicity, is compatible with a pharmaceutical use.

In particular, by virtue of such properties, and especially the increased magnetic response thereof, the maximum synergy is obtained with the magnetic handpiece of the present invention. In other words, when the ferromagnetic agent is an aqueous suspension of carbonyl iron as defined above, the magnetic handpiece according to the present invention provides the best performance thereof in terms of anchoring the head of the endoscopic guide.

Describing now the magnetic handpiece system according to the invention, it, indicated as whole by reference numeral 1, comprises a magnetic handpiece 2 and a casing 3.

The magnetic handpiece 2 comprises a magnetic field source configured to apply a retaining force to the ferromagnetic agent, with respect to a transverse pulling direction, which is greater than 5 Newton at a distance between 2 centimeters and 10 centimeters.

The term “apply a retaining force to the ferromagnetic agent with respect to a transverse pulling direction” means the retaining force to the ferromagnetic agent with respect to a pulling substantially perpendicular to the direction of maximum attraction between magnet and ferromagnetic body, as determined by the lines of force of the magnetic field.

The retaining force of the expandable container against the wall of the colon, with respect to a transverse pulling direction which, as mentioned, is to be equal to or greater than 2 Newton, or equal to or greater than 5 Newton, to allow adequate pulling of the endoscopic guide, depends both on the amount of ferromagnetic agent in the container and on the magnetic field applied, which in turn depends on the distance at which the source of the magnetic field is from the container filled with the ferromagnetic agent. Usually, such a distance is between 1 and 5 cm in most colon segments. However, such a distance is greater in obese patients, for example up to 10 cm.

The magnetic field source according to the invention consists of one or more permanent magnets. For the purposes of the invention, it is possible to use a magnetic field equal to that generated by a permanent magnet with discoidal magnetization N52 (i.e., a magnet in which the maximum magnetic energy per volume storable in the magnet is 52 Mega-Gauss-Oersted) having diameter between 50 and 100 mm, thickness between 30 and 80 mm, and weight between 1.8 and 4.5 kg. It can be made of sintered neodymium, sintered ferrite, plasto-neodymium, or the like.

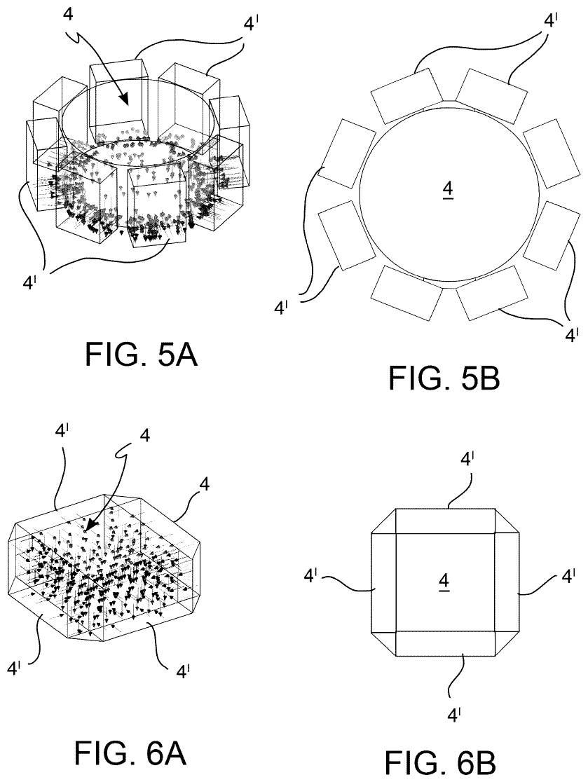

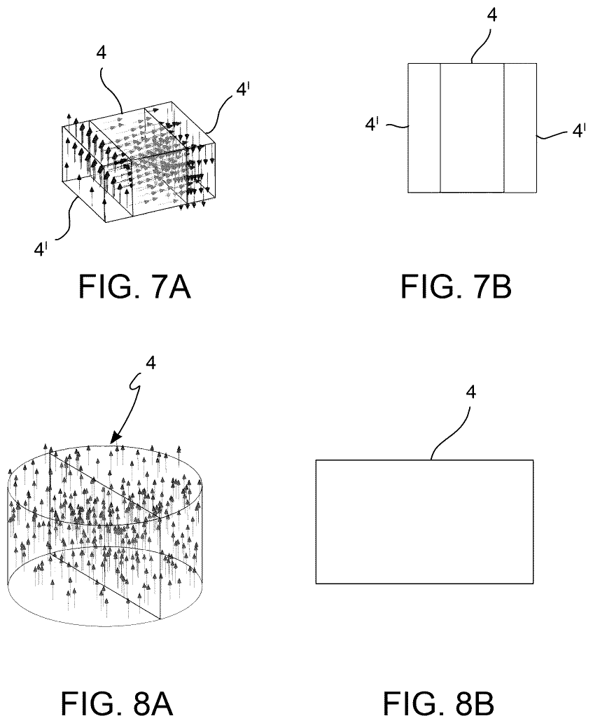

In particular, by way of example, the following magnetic source configurations can be used (FIGS. 5A-8B):
1) a Halbach Array type configuration comprising a plurality of magnets 4′, arranged in a crown about a cylinder-shaped central magnet 4, preferably 8 magnets 4′ arranged in a crown (FIGS. 5A and 5B);
2) a Halbach Array-type configuration comprising four magnets 4′ arranged along the sides of a parallelepiped central magnet 4, i.e., peripheral with respect to said central magnet 4 (FIGS. 6A and 6B);
3) a Halbach Array-type configuration comprising two magnets 4′ arranged sandwiched along two sides of a parallelepiped central magnet 4 (FIGS. 7A and 7B);
4) a cylinder-shaped magnet 4 (FIGS. 8A and 8B).

FIGS. 5a, 6A, 7A and 8A also show the direction of the magnetic field by means of the arrows.

In particular:
in configuration 1), the central magnet 4 is placed with axial polarization, while the plurality of magnets 4′ in a crown has a polarization arranged along the rays converging towards the longitudinal axis of the central magnet 4;
in configuration 2), the central magnet 4 is placed with axial magnetization, while the four peripheral magnets 4′ have a magnetization parallel to a plane perpendicular to the magnetization direction of the central magnet 4;
in configuration 3), the central magnet 4 and the two sandwiched magnets 4 are placed with magnetization along directions at 90° from each other:
in configuration 4), magnet 4 has axial magnetization.

In preferred embodiments, the magnetic source according to configuration 1) consists of a central magnet 4 having a diameter of about 74 mm and a thickness of about 35 mm, and eight parallelepiped magnets 4′ having dimensions of about Lc15×Lu26×Sp35 mm (where Lc=short side; Lu=long side; Sp=thickness), where the total weight of the magnetic source is about 2 kg, and where the eight magnets 4′ in a crown are spaced apart from the central magnet 4 by about 1 mm. This latter feature allows optimizing the capacity of magnetic attraction in relation to the weight of the magnet.

In preferred embodiments, the magnetic source according to configuration 2) consists of a central magnet 4 having dimensions of L60-61 mm×Sp35-44 mm, and four peripheral magnets 4′ having dimensions of Lc15-16×Lu60-61×Sp35-44 mm, where the total weight of the magnetic source is between 2 and 2.5 kg.

In preferred embodiments, the magnetic source according to configuration 3) consists of a central magnet 4 having dimensions of about Lc44×Lu45×Sp58 mm, and two sandwiched magnets 4′ having dimensions of about Lc28-16×Lu45×Sp58 mm, where the total weight of the magnetic source is about 2 kg.

In preferred embodiments, the magnetic source according to configuration 4) consists of a magnet 4 having dimensions of about D85-86 mm×Sp45-45.5 mm (where D is the diameter of the base of the cylinder), where the total weight of the magnetic source is about 2 kg.

In preferred embodiments, the step of applying the magnetic field can be performed by applying such a magnetic field in a progressive manner. If indeed the required magnetic field were applied quite quickly, it would cause a sudden movement of container 4, which would then cause a sudden and painful stretching of the patient's viscera. The progressive application of the magnetic field can be obtained in two ways:
A) increasing the magnetic field applied by progressively varying the distance of the external magnetic field source, or
B) progressively introducing aliquots of ferromagnetic agent into the expandable container 4 with external magnetic field already applied.

The magnetic field source is contained in a handpiece especially adapted for this application, which is an object of the present invention and which will be described below.

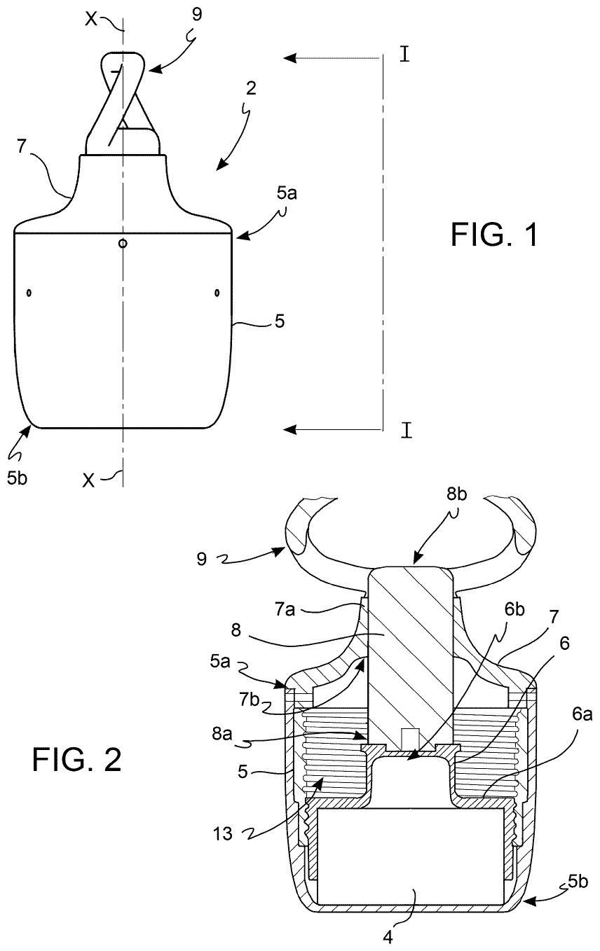

The magnetic handpiece 2, shown in FIGS. 1-4, comprises a hollow body 5 having a longitudinal axis X-X, a first end 5a and a second end 5b, in which a movable support element 6 of the magnetic source is movably accommodated through a screw coupling, where the movable support element 6 is movable along axis X-X between a raised, or non-operating, position and a plurality of lowered operating positions.

In other words, the movable support element 6 can be placed in an operating position adjacent to the second end 5b of body 5 (where the maximum magnetic attractive force is obtained on the support surface of handpiece 2) or in a plurality of intermediate operating positions between the first end 5a and the second end 5b of body 5.

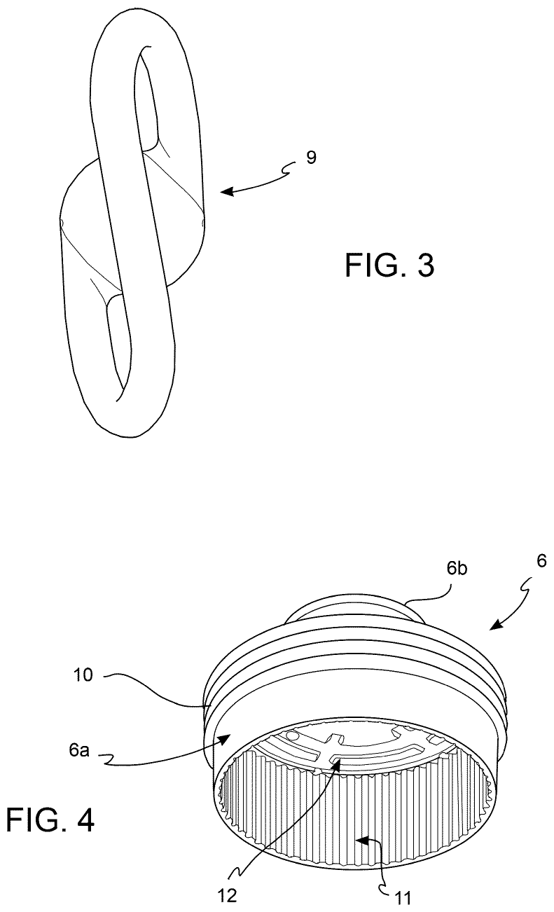

Body 5 comprises a closing element 7 at the top which comprises a central sleeve portion 7a having an axial opening 7b in which a shaft 8 is inserted. Shaft 8 is connected at an inner end 8a to the movable support element 6 and at an outer end 8b to a handle 9. In certain embodiments, as shown in FIGS. 1 and 3, handle 9 has a spiral shape, for example in clockwise direction, to make the correct rotation direction more intuitive for screwing and unscrewing the movable support element 6 and thus increasing or decreasing the magnetic attraction force applied.

Body 5 and the closing element 7 can be made in two separate parts, as shown in the drawings, but they can also be made in a single piece.

The movable support element 6, shown in FIG. 4, comprises a hollow cylindrical portion 6a and a sleeve portion 6b in which shaft 8 is accommodated and fastened.

The hollow cylindrical portion 6a comprises an outer thread 10, while the inner surface comprises a plurality of ribs 11, the function of which is to increase the contact surface with the magnet.

The hollow interior 12 is configured to house the magnetic source which can be a single magnet 4 or one of the Halbach configurations described above, for example.

Body 5 comprises in turn, on an inner surface thereof, a thread 13 which is couplable to thread 10 of the movable support element 6.

Thereby, by rotating shaft 8 by means of handle 9, the movable support element 6 can be screwed to or unscrewed from the inner thread 13 of body 5, which results in the magnetic source moving close to or away from (or vice versa, depending on the direction of the thread) the second end 5b of body 5.

The shaft is provided with a graduated scale which indicates the intensity level of the selected magnetic field after the handle has been turned. By applying the scale to the shaft, it is more visible as well as more convenient from a construction viewpoint. The height of the shaft decreases as handle 9 is rotated and therefore the magnet is lowered. This phenomenon makes more apparent the magnet level selected and therefore mitigates the risk of possible errors of adjusting the intensity of the magnetic field.

The outer shell of the handpiece could be entirely or partially transparent to facilitate viewing the magnet or to facilitate inspecting the patient's skin on which the handpiece is placed. The latter point can be advantageous for identifying possible phenomena of transillumination.

Transillumination means that situation in which the light of the colonoscope is visible externally through the patient's skin. Such a phenomenon is a sign that the abdominal wall is thin and also allows accurately identifying where the endoscope tip is with respect to the abdominal surface.

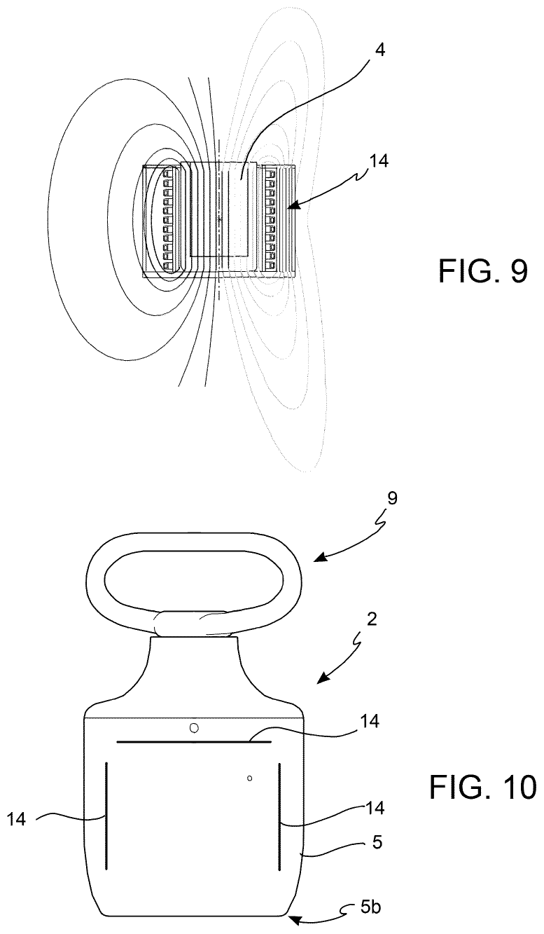

In certain embodiments, the magnetic handpiece 2 comprises magnetic field concentrator elements 14 (diagrammatically shown in FIG. 10). The magnetic field concentrator elements 14 are made by ferromagnetic material shields arranged above the magnetic source and along the walls of the movable support element 6 or body 5, for example in the form of a plurality of plates or as a continuous or discontinuous annular band. In other embodiments, the magnetic field concentrator elements 14 are only placed along the walls of the movable support element 6 or body 5.

The magnetic field concentrator elements 14 can be embedded in the material from which the body 5 or the movable support element 6 is made or they can be arranged along a surface thereof.

The magnetic field concentrator elements 14 can consist of a single sheet of ferromagnetic material or a plurality of sheets, placed in contact or spaced apart.

FIG. 9 shows, by way of example, how the magnetic field is distorted by a magnetic field concentrator element 14 (on the right) with respect to an unshielded magnetic field (on the left). As can be seen, the presence of the magnetic field concentrator elements 14 allows reducing the expansion of the field lines in transverse directions and concentrating them in the operating direction of the magnetic handpiece 2. Thereby, the magnetic attraction, and therefore the efficiency of the magnetic source, towards the balloon filled with ferromagnetic material inserted in the organ to be analyzed is increased, while the possible interference with nearby devices or objects is reduced.

The efficiency of the shielding depends on the magnetic permeability of the material with which the magnetic field concentrator elements 14 are made. A preferred material is a metal alloy comprising 76-80%, or about 80%, nickel, about 14.5-15% iron, 3-6%, or about 5%, molybdenum, 4-55% copper, and traces of silicon. Such an alloy is marketed, for example, under the trade names Mumetal, Cryoperm, Vacoperm and Ultraperm.

The invention further relates to a magnetic handpiece system 1 comprising a magnetic handpiece 2 as described above and a casing 3 of said magnetic handpiece 2.

Casing 3 is an important element because it must allow the safe storing of the magnetic handpiece 2 when it is not in use and a quick and easy removal and reinsertion during the operating step.

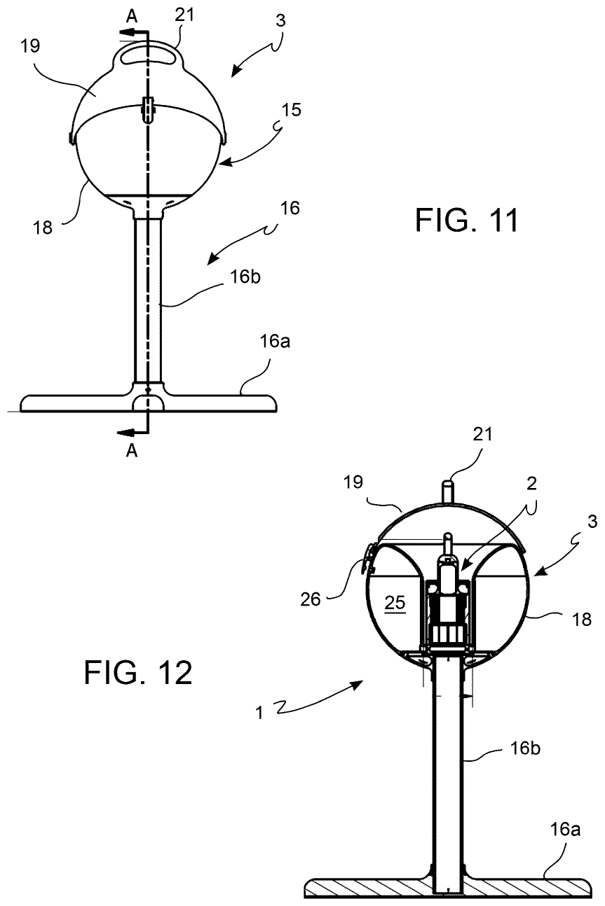

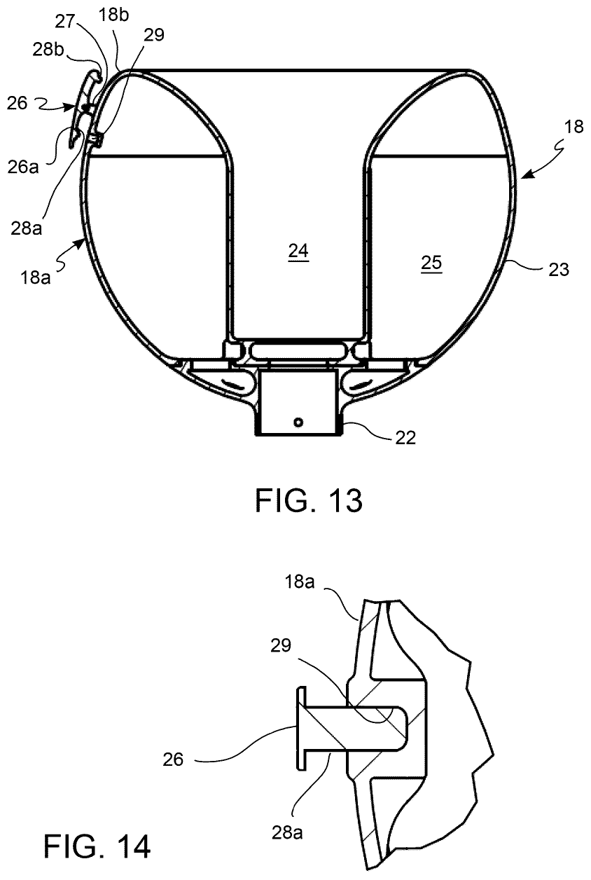

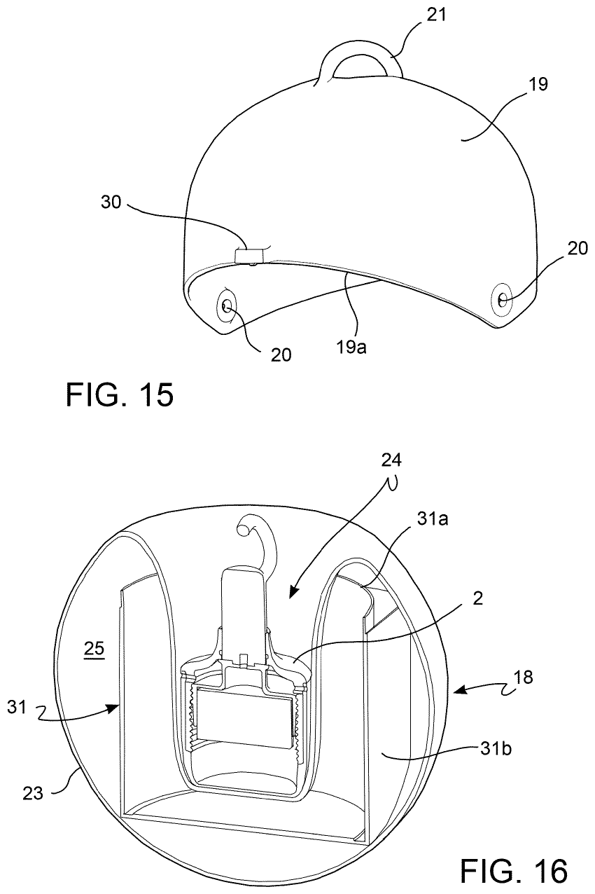

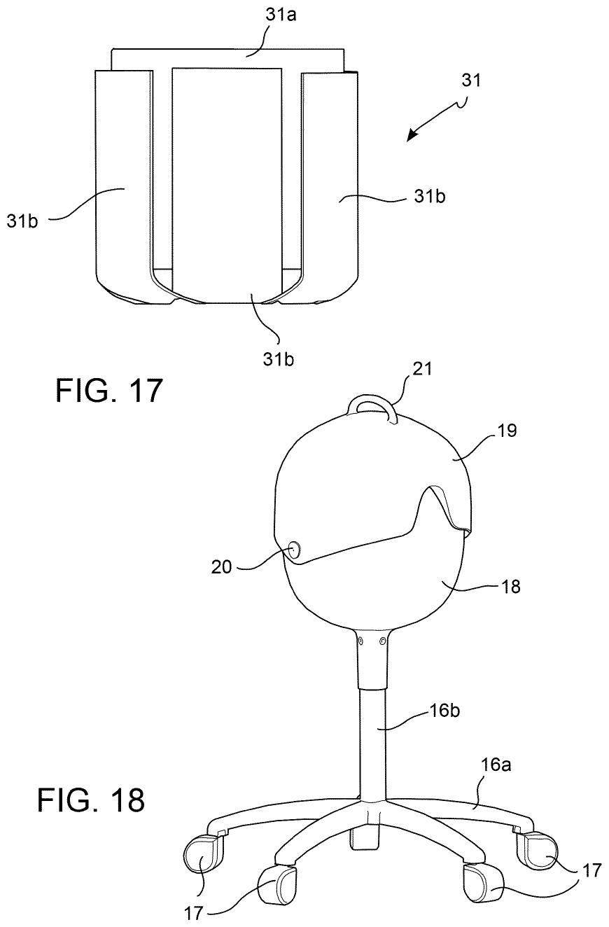

The casing 3, shown in FIGS. 11-18, comprises a containment shell 15 placed on a stand 16 comprising a base 16a and a pole 16b. As shown in FIG. 18, the base 16a can preferably comprise wheels 17 for allowing the operator to easily move the casing 3.

The height of stand 16 is such that the handpiece 2 contained in casing 3 can be easily gripped by any operator without the need for the operator to bend down considerably to take it. However, the height of stand 16 was decreased as much as possible to lower the center of gravity of the device and limit the risk of tipping. Moreover, if the carriage is low, handpiece 2 is quite visible and grippable from any side.

The containment shell 15 consists of a basket 18 and a lid 19 hinged to basket 18. Advantageously, basket 18 and lid 19 are shaped like a spherical cap such that the containment shell 15, as a whole, is substantially spherical in shape. Lid 19 comprises two hinges 20 placed in diametrically opposite points. Thereby, the opening and closing of lid 19 can occur by moving the related spherical cap onto that of basket 18 so as to be easily operable with one hand alone. This further allows limiting the overall dimensions of the lid once it is opened and, as compared to a conventional lid with a hinge at the end, avoiding for possible knocks to result in the accidental closing of the lid. If overturned, the conventional lid requires space for the overturning thereof. If opened, by only 90 degrees, for example, the operator can have limited access to the handpiece. Instead, the spherical lid allows the handpiece to be gripped from any position. Finally, once opened, the spherical lid is kept open by the force of gravity and there is no risk of accidental closing if the carriage is knocked.

For this purpose, lid 19 comprises a handle 21 which facilitates the movement of the lid (when the lid is released) and the carriage (when the lid is closed and locked).

Moreover, the lid is provided with shielding close to the inner surface. Such a shielding is ferromagnetic and could attract the magnet when the handpiece is removed. In order to limit this phenomenon, the present lid was designed so that the lower surface of the lid always remains hidden and the upper surface is spaced apart from the screen by 4-5 cm. Such a distancing ensures that the magnetic attraction is weak if the magnet approaches, and therefore avoids accidental blows of the handpiece on the lid. Such blows could result in damage to the device or harm to the operator if his/her hands were in the way. Also the screen in the casing was designed so as to be spaced apart from the outer surface by 4-5 cm in order to limit the magnetic attraction force between the handpiece and the shielding.

In certain embodiments, basket 18 is semi-ovoidal in shape and lid 19 is shaped like a spherical cap.

Basket 18 comprises a sleeve 22 at the bottom for the connection with pole 16b and has a toroidal body 23 which is preferably internally empty so as to create a space 25. Thereby, the structure can be made lighter.

The toroidal body 23 comprises a central seat 24, which, as shown in FIGS. 12 and 16, is sized to house the magnetic handpiece 2. The central seat 24 is conical in shape to facilitate the repositioning and centering of handpiece 2, also limiting the risk of accidental blows of handpiece 2 during the repositioning in the casing.

The central seat 24 is such as to always keep handpiece 2 vertical. This allows the following advantages.

The magnetic field on the sides of the handpiece is weaker and less extended in space. The operator has the handpiece in a comfortable position for gripping it and in the correct position used during the endoscopic procedure. The casing prevents hand access to the body of handpiece which is the component containing the magnet, and therefore at the risk of causing accidents involving hands being crushed. Therefore, the casing promotes the correct gripping of the handpiece by the operator by gripping the handle or the shaft alone.

A catch 26 comprising a tilting bar 26a hinged in a median point thereof on a ridge 27 placed on surface 18a is placed on the outer surface 18a of basket 18, in a position close the upper edge 18b. A first and a second retainer tooth 28a, 28b are placed close to the two ends of bar 26a. The first retainer tooth 28a is configured to snappingly couple in a seat 29 recessed in the surface 18a of basket 18, while the second retainer tooth 28b is configured to be coupled to a ridge 30 placed along the edge 19a of lid 19, in a median position with respect to the hinges 20. Catch 26 is designed to be handled by a single hand, obtaining two stop positions: one corresponding to an opening position of lid 19 so that catch 26 does not obstruct the opening thereof; the other corresponding to a closing position, keeping lid 19 closed on basket 18.

In preferred embodiments, as shown in FIG. 16, basket 18 comprises magnetic field shielding elements 31. When the magnetic handpiece 2 is stored in casing 3, the shielding elements 31 prevent (or at least reduce the risk that) the magnetic field thereof to expand outwards, being capable of interfering with externally located apparatuses.

The shielding elements 31 consist of ferromagnetic material plates, preferably the above-described metal alloy comprising 76-80%, or about 80%, nickel, about 14.5-15% iron, 3-6%, or about 5%, molybdenum, 4-55% copper, and traces of silicon.

In particular embodiments, the shielding elements 31 comprise an annular sheet 31a and a plurality of U-shaped sheets 31b configured so as to also cover the bottom of basket 18.

Lid 19 has a total surface which is sufficiently extended so as to contain the shielding elements 31 and ensure a spacing of at least 4-5 cm not only from the upper surface, but also from the edge.

In order to ensure lid 19 can be completely opened and leave the entire central seat 24 of handpiece 2 accessible from all directions, lid 19 was recessed in the back portion thereof (that opposite to catch 26) so that the axis of the casing does not prevent the complete opening of lid 19.

The shielding elements 31 are preferably accommodated in space 25 inside the toroidal body 23.

A silicone pad for limiting the impact of handpiece 2 when it is resting or when the casing is moved, is on the bottom of the handpiece housing. The pad is only resting on the bottom so that it is easy to extract and clean.

The overall thickness of the shielding elements 31 is preferably between 3 mm and 6 mm. The conical shape of the central seat 24 therefore promotes the centering of handpiece 2 also with respect to the shielding of the casing and the outer surface of the carriage. Thereby, the extension of the magnetic field into the external environment can be limited.

The invention further relates to an endoscopy kit, comprising a magnetic handpiece 2 according to the invention, an endoscopic guide as described in EP3399901A1, a pre-filled syringe filled with an aqueous suspension of a ferromagnetic agent in a preset amount, and optionally a catheter.

In certain embodiments of the kit of the invention, the syringe is preloaded with a suspension of said ferromagnetic agent, preferably in a volume between 20 and 30 ml, more preferably about 25 ml, and where said ferromagnetic agent is carbonyl iron.

In alternative embodiments, the kit according to the invention comprises:
a glass vial filled with the powdered ferromagnetic agent, preferably carbonyl iron, and a pre-filled syringe filled with saline solution (aqueous solution of NaCl and/or sodium citrate), or
a glass vial with the ferromagnetic agent suspension, preferably carbonyl iron, and an empty syringe.

Obviously, the endoscopy system according to the present invention can be operated in an entirely similar manner to the endoscopy system described in EP3399901A1 according to the method described above, which is a further object of the present invention.

In essence, it has been found how the invention achieves the preset tasks and objects because it allows providing a magnetic handpiece 2 which allows obtaining a strong anchoring, which is mainly the result of the synergy obtainable by virtue of the considerable amount of ferromagnetic agent which can be introduced into the expandable container of the endoscopic guide and the increased magnetic field applicable from the outside by said magnetic handpiece 2. Said synergy is particularly apparent when the ferromagnetic agent is carbonyl iron.

The magnetic handpiece system 1 according to the invention is also easy and affordable to make, as well as quick and safe to use. In particular, casing 3 can be opened and closed with one hand alone, leaving free the operator's other hand. The force of attraction of the magnetic handpiece 2 is easy to adjust by rotating handle 9 in clockwise or counter-clockwise direction so as to take the magnetic field source close to or away from (or vice versa, according to the direction of the thread) the surface of the patient's abdomen, respectively. The shielding of the casing and the magnetic field concentrator elements of the magnetic handpiece allow reducing magnetic interactions with objects or devices in directions other than those in use.

It is apparent that only some particular embodiments of the present invention have been described, to which those skilled in the art will be able to make all changes required to adapt it to particular applications, without departing from the scope of protection of the present invention.

In practice, the materials used, as long as they are compatible with the specific use, as well as the contingent dimensions and shapes, can be varied according to the requirements and advancement of the art.

1. A magnetic handpiece system, comprising a magnetic handpiece and a casing, wherein the magnetic handpiece comprises a magnetic field source comprising one or more permanent magnets and wherein the magnetic handpiece comprises a hollow body having a longitudinal axis, a first end and a second end, in which a movable support element of the magnetic source is movably accommodated through a screw coupling, wherein the movable support element is movable along the longitudinal axis between a raised, or non-operating, position and a plurality of lowered operating positions.

2. The system according to claim 1, wherein the body comprises a closing element at a top which comprises a central sleeve portion having an axial opening in which a shaft is inserted, an inner end of the shaft being connected to the movable support element and an outer end of the shaft being connected to a handle.

3. The system according to claim 1, wherein the movable support element comprises a hollow cylindrical portion comprising an outer thread and an inner surface comprising a plurality of ribs, the hollow cylindrical portion defining a hollow interior configured to house the magnetic source; and wherein the body comprises, on an inner surface thereof, a thread which is couplable to the thread of the movable support element.

4. The system according to claim 1, wherein the magnetic source has a magnetization equivalent to a discoidal permanent magnet with N52 magnetization having a diameter between 50 and 100 mm, a thickness between 30 and 80 mm, and a weight between 1.8 and 4.5 kg, and is selected from:
a) a cylinder-shaped magnet having axial magnetization;
b) a Halbach Array type configuration comprising a plurality of magnets, arranged in a crown about a cylinder-shaped central magnet;
c) a Halbach Array-type configuration comprising four magnets arranged along sides of a parallelepiped central magnet, being peripheral with respect to said central magnet;
d) a Halbach Array-type configuration comprising two magnets arranged in a sandwich along two sides of a parallelepiped central magnet,
wherein:
in configuration b), the central magnet is placed with axial polarization, while the plurality of magnets in a crown has a polarization arranged along rays converging towards the longitudinal axis of the central magnet,
in configuration c), the central magnet is placed with axial magnetization, while the four peripheral magnets have a magnetization parallel to a plane perpendicular to a magnetization direction of the central magnet,
in configuration d), the central magnet and the two sandwiched magnets are placed with magnetization along directions at 90° from each other.

5. The system according to claim 4, wherein:
the magnetic source according to configuration a) consists of a magnet having dimensions of about D85-86 mm×Sp45-45.5 mm, wherein the total weight of the magnetic source is about 2 kg;
the magnetic source according to configuration b) consists of a central magnet having a diameter of about 74 mm and a thickness of about 35 mm, and of eight parallelepiped magnets having dimensions of about Lc15×Lu26×Sp35 mm, wherein a total weight of the magnetic source is about 2 kg and wherein the eight magnets in a crown are spaced apart from the central magnet by about 1 mm;
the magnetic source according to configuration c) consists of a central magnet having dimensions of Lc=Lu60-61 mm×Sp35-44 mm, and of four peripheral magnets having dimensions of Lc15-16×Lu60-61×Sp35-44 mm, wherein the total weight of the magnetic source is between 2 and 2.5 kg;
the magnetic source according to configuration d) consists of a central magnet having dimensions of about Lc44×Lu45×Sp58 mm, and of two sandwiched magnets having dimensions of about Lc28-16×Lu45×Sp58 mm, wherein the total weight of the magnetic source is about 2 kg,
where Lc=short side, Lu=long side, Sp=thickness, and D is the diameter of a base of the cylinder.

6. The system according to claim 1, wherein the magnetic handpiece comprises magnetic field concentrator elements comprising ferromagnetic material shields arranged along walls of the movable support element or the body, in the form of a plurality of plates or as a continuous or discontinuous annular band, and comprising a single sheet of ferromagnetic material or a plurality of sheets, placed in contact or spaced apart.

7. The system according to claim 6, wherein said ferromagnetic material is a metal alloy comprising 76-80%, nickel, about 14.5-15% iron, 3-6%, 4-55% copper, and traces of silicon.

8. The system according to any one of claim 1, wherein the casing comprises a containment shell placed on a stand comprising a base and a pole, the containment shell comprising a basket and a lid comprising a handle, the lid being hinged on the basket at two hinges placed in diametrically opposite points and wherein the lid and optionally the basket as well are shaped like a spherical cap so that the containment shell, as a whole, is substantially spherical or semi-ovoidal in shape.

9. The system according to claim 8, wherein the basket comprises a toroidal body which is preferably internally empty to create a space, the toroidal body comprising a central seat sized to house the magnetic handpiece.

10. The system according to claim 8, wherein a catch comprising a tilting bar hinged in a median point thereof on a ridge placed on the surface is placed on an outer surface of the basket, in a position proximate an upper edge, a first and a second retainer tooth being placed proximate two ends of the bar, wherein the first retainer tooth is configured to snappingly couple in a seat recessed in the surface of the basket, while the second retainer tooth is configured to be coupled to a ridge placed along an edge of the lid, in a median position with respect to the hinges.

11. The system according to any one of claim 8, wherein the basket comprises magnetic field shielding elements comprising ferromagnetic material plates.

12. The system according to claim 11, wherein the shielding elements comprise an annular sheet and a plurality of U-shaped sheets configured to also cover a bottom of the basket, the shielding elements being accommodated in a space inside the toroidal body.

13. A magnetic handpiece as defined in claim 1.

14. A casing for a magnetic handpiece as defined in claim 8.

15. An endoscopy kit, comprising:
a magnetic handpiece as defined in claim 1,
an endoscopic guide comprising a tubular guide element and an anchoring head, wherein the tubular guide element comprises a longitudinal cavity, the anchoring head comprising an expandable container configured to accommodate a ferromagnetic agent,
a pre-filled syringe filled with an aqueous suspension of a preset amount of ferromagnetic agent; or a glass vial filled with the powdered ferromagnetic agent, and a pre-filled syringe filled with saline; or a glass vial with the ferromagnetic agent suspension, and an empty syringe.

16. The system according to claim 1, wherein the body comprises a closing element at a top which comprises a central sleeve portion having an axial opening in which a shaft is inserted, an inner end of the shaft being connected to the movable support element and an outer end of the shaft being connected to a handle having a spiral shape.

17. The system according to claim 1, wherein the body comprises a closing element at a top which comprises a central sleeve portion having an axial opening in which a shaft is inserted, an inner end of the shaft being connected to the movable support element and an outer end of the shaft being connected to a handle having a spiral shape in a clockwise direction, to make a correct rotation direction more intuitive for screwing and unscrewing the movable support element and increasing or decreasing the magnetic attraction force applied.

18. The system according to claim 1, wherein the magnetic handpiece comprises magnetic field concentrator elements comprising ferromagnetic material shields arranged along walls of the movable support element or the body and above the magnetic source, in the form of a plurality of plates or as a continuous or discontinuous annular band, and comprising a single sheet of ferromagnetic material or a plurality of sheets, placed in contact or spaced apart.

19. The system according to claim 8, wherein the basket comprises magnetic field shielding elements comprising ferromagnetic material plates, of a metal alloy comprising 76-80%, nickel, about 14.5-15% iron, 3-6%, molybdenum, 4-55% copper, and traces of silicon, the overall thickness of the shielding elements being between 3 mm and 6 mm.

20. An endoscopy kit, comprising:
a magnetic handpiece as defined in claim 1,
an endoscopic guide comprising a tubular guide element and an anchoring head, wherein the tubular guide element comprises a longitudinal cavity, the anchoring head comprising an expandable container configured to accommodate a ferromagnetic agent,
a pre-filled syringe filled with an aqueous suspension of a preset amount of ferromagnetic agent; or a glass vial filled with the powdered ferromagnetic agent comprising carbonyl iron, and a pre-filled syringe filled with saline comprising an aqueous solution of NaCl and/or sodium citrate; or a glass vial with the ferromagnetic agent suspension comprising carbonyl iron, and an empty syringe.
